(19) United States
(12) Patent Application Publication
(10) Pub. No.: US 2009/0068263 A1
(43) Pub. Date: Mar. 12, 2009

(54) MULTIPLE UNIT COMPOSITIONS

(75) Inventors: Amit Krishna Antarkar, Nagpur (IN); Sunil Beharilal Jaiswal, Nagpur (IN); Maya Janak Shah, Mumbai (IN); Abdul Shajahan, Madurai (IN)

Correspondence Address: LAHIVE & COCKFIELD, LLP;FLOOR 30, SUITE 3000, ONE POST OFFICE SQUARE, BOSTON, MA, 02109, US

(73) Assignee: Themis Laboratories Private Limited, Mumbai , Maharashtra (IN)

(21) Appl. No.: 12/297,753

(22) PCT Filed: Apr. 16, 2007
(86) PCT No.: PCT/IB2007/001029

§ 371 Date: Oct. 20, 2008

(30) Foreign Application Priority Data
Apr. 20, 2006 (IN) 627/MUM/2006

(51) Int. Cl. A61K 9/26; A61K 9/14; A61K 31/435

(52) U.S. Cl. 424/470; 424/497; 514/338

(57) ABSTRACT

The present invention provides a multiple unit compositions comprising of enteric coated pellets and at least one tablet excipient, wherein each pellet comprises: i) a core comprising active ingredient(s); ii) optionally a separating layer coated on the core; iii) at least two enteric layers comprising of enteric polymers and plasticizer either coated on the core or on the separating layer to obtain enteric coated pellets, such that the last enteric layer is formed from a solution comprising of enteric polymer and plasticizer in organic solvent(s), resulting in no appreciable change in release profile of active ingredient on compression of enteric coated pellets into tablets.

### FIELD OF THE INVENTION

The invention relates to multiple unit tablet compositions comprising enteric coated pellets and process for preparation thereof.

### BACKGROUND OF THE INVENTION

Multiple unit particulate system (MUPS) has several distinct advantages over single unit system such as
1. Multiple unit particles get distributed throughout the GI tract thereby avoiding localized accumulation and local irritation.
2. Minimal inter and intra subject variation.
3. Incorporation of 2 or more incompatible drugs in a single dosage form.
4. Allows preparation of multi-dose formulation without any change in process or formulation.
5. Particles having different release profiles being delivered simultaneously.

Though MUPS is the delivery system of choice, it needs to be formulated in a single unit for ease of administration. However, there are certain categories of active ingredients that are unstable in gastric media or cause gastric irritation which need to be protected by an enteric coating.

Drugs belonging to such categories include omeprazole, pantoprazole, lansoprazole, rabeprazole, substituted phenylmethylsulfinyl-1H-benzimidazoles, cycloheptapyridin-9-ylsulfinyl-1H-benzimidazoles or pyridin-2-ylmethylsulfinylthieno-imidazoles, leminoprazole, 2-(4-methoxy-6,7,8,9-tetrahydro-5H-cyclohepta[b]pyridin-9-ylsulfinyl)-1H-benzimidazole (nepaprazole) and duloxetine or their pharmaceutically acceptable salts such as rabeprazole sodium or their enantiomers such as esomeprazole or pharmaceutically acceptable salts of their enantiomers such as esomeprazole magnesium trihydrate.

Enteric coated multiple unit particulate systems are generally prepared by coating the particles containing drug with enteric polymer that delay release of the drug. When these enteric coated multiple unit particles are compressed into tablets, the polymeric film gets ruptured, exposing the drug molecules to gastric contents, resulting in destruction of acid labile drug molecules.

PCT publication WO03/103637 teaches modified release multiple unit drug delivery system wherein each unit comprises inert core coated with first coating layer that includes one or more drugs and one or more rate controlling polymers and an outer layer comprising a material that is elastic and/or compressible waxy material such as PEG. The rate controlling membrane controls release of the drug over a period of 24 hours. This waxy outer layer protects the release control polymer layer from cracking during compression. The process requires an additional coating with waxy material (PEG) making the product relatively more expensive.

EP 1072257 discloses a sustained release polymer incorporated multiple-unit sustained release tablet consisting of a granular part and a powdery part. The granular part comprises a matrix of water insoluble polymer ethyl cellulose of viscosity more than 15 cps and an active ingredient. These matrix granules are further coated with water insoluble release controlling polymer that controls release of the drug over 24 hours. This coated matrix granules are blended with powdery part in the ratio of 1:0.5 or more to ensure disintegration into sub units. Though such compositions solve the problems related to breakdown of coating when compressed into tablets, it fails to prevent the drug from being appreciably released in acidic medium.

PCT publication WO03/103637 and EP 1072257 do not teach methods to prevent the release of the drug in the acidic pH and to provide immediate release of the drug in near neutral to alkaline pH.

U.S. Pat. No. 6,328,994 discloses orally disintegratable tablets comprising lansoprazole granules having superior acid resistance property after compression. The process necessarily requires lansoprazole core to be coated with an enteric coating agent and a sustained release agent to obtain lansoprazole fine granules. An essential requirement is that the average particle diameter of the granules should be less than 400 microns for tablets to retain acid resistance property after compression.

WO 97/25029 discloses a preparation of disintegratable tablets comprising three types of pellets viz. active pellets, deformable pellets and disintegratable pellets. The drug pellets are coated with a controlled release binder, soft pellets contain an ester of fatty acid and the disintegratable pellets preferably comprise of a water insoluble inorganic powder. The soft pellets deform during the tabletting process that minimize damage to drug pellets while the disintegratable pellets ensure retention of release characteristic from drug pellets. The process is cumbersome as it requires preparation of 3 different types of pellets.

U.S. Pat. No. 6,923,984 discloses use of biologically inactive cushioning beads of 0.5 to 2 mm to protect brittle coating over active beads during compression. The biologically inactive cushioning beads comprise at least one compressible cushioning component and optionally another biologically inactive compressible cushioning component or pharmaceutically acceptable excipient. The compressible cushioning component essentially consists of a microcrystalline hydrocarbon wax or a natural wax at least 30% by weight of the biologically inactive cushioning beads. The productivity of the process is low as it requires preparation of 2 types of beads.

U.S. Pat. No. 5,817,338, U.S. Pat. No. 5,753,265, EP0723436 and EP0723437 disclose oral pharmaceutical multiple unit tablet compositions comprising at least one tablet excipient and multiple of pellets or granules comprising acid-labile omeprazole. The pellets or granules are covered with at least one enteric coating layer comprising a plasticizing compound. An essential requirement is that the plasticizing compound should be more than 20% but less than 50% by weight of the enteric coating polymer to retain acid resistance property after compression.

Disclosures in prior art to protect the desired layer from cracking during compression involve the use of:
1) Elastic and compressible outer layer comprising waxy materials such as PEG to protect inner rate controlling polymer layer, or
2) Powdery part to granular part at least in the ratio of 0.5:1, or
3) Sustained release agent in combination with enteric coating agent, or
4) Soft pellets or deformable pellets or biologically inactive cushioning beads, or
5) Plasticizer of more than 20% w/w of enteric polymer in enteric layer.

There is a long standing need to provide patient compliant tablet dosage forms comprising multiple unit pellets that are appropriately protected such that there is no appreciable change in the release profile of acid labile active ingredient on compression of enteric coated pellets into tablets and to ensure that the acid labile drug is prevented from being released in the gastric region.

### OBJECTS OF THE INVENTION

The main object of the present invention is to provide compressible composition comprising enteric coated multiple unit particles comprising active ingredient; and a tablet excipient such that there is no appreciable change in the release profile of active ingredient before and after compression, and that the drug is not released in the gastric region.

Another object of the invention is to provide a process for the preparation of such compressible composition comprising enteric coated multiple unit particles comprising active ingredient and a tablet excipient.

It is yet another object of the invention to provide orally dispersible tablets comprising enteric coated multiple unit particles comprising active ingredient; and a tablet excipient such that there is no appreciable change in the release profile of active ingredient before and after compression and process for their preparation.

### SUMMARY OF INVENTION

The present invention provides a multiple unit tablet composition comprising of enteric coated pellets and at least one tablet excipient, wherein each pellet comprises:
i) a core comprising active ingredient(s);
ii) optionally a separating layer coated on the core;
iii) at least two enteric layers comprising of enteric polymers and plasticizer either coated on the core or on the separating layer to obtain enteric coated pellets, such that the last enteric layer is formed from a solution comprising of enteric polymer and plasticizer in organic solvent(s); the total enteric polymers being at least 20% by weight of the enteric coated pellets and plasticizer up to 15% by weight of enteric polymers, resulting in no appreciable change in release profile of active ingredient on compression of enteric coated pellets into tablets.

The enteric coated pellets comprise of two or more enteric layers, which differ in composition and ratio.

The total enteric polymers are at least 20%, preferably 30% to 70%, more preferably 40% to 60% by weight of the enteric coated pellets.

In one aspect of the invention, the enteric coated pellets have two enteric layers, the ratio of enteric polymer in these two layers is 0.8:0.2 to 0.2:0.8. It is preferred to have enteric polymer or polymers in the range of 0.7: to 0.3 to 0.3:0.7 and more preferably from 0.6:0.4 to 0.4:0.6.

In another aspect of the invention, the enteric coated pellets have three or more enteric layers, the enteric polymer(s) in one layer is at least 10% by weight of the total enteric polymers.

Plasticizers in the enteric layers is up to 15%, preferably up to 12.5%, more preferably up to 10% by weight of enteric polymer.

The tablet of the present invention may be in the form of swallowable tablet or orally dispersible tablet.

The present invention also provides a process for the preparation of multiple unit compositions comprising of enteric coated pellets, exhibiting no appreciable change in release profile of active ingredient on compression, comprising steps:
i) preparation of a core comprising active ingredient(s);
ii) optionally creating a separating layer on the core;
iii) coating core or separating layer coated core with at least two enteric layers comprising of enteric polymers and plasticizer to obtain enteric coated pellets, wherein the enteric polymers is at least 20% by weight of the enteric coated pellets and plasticizer is up to 15% by weight of enteric polymers, wherein last enteric layer is formed from a solution comprising of enteric polymer(s) and plasticizer in organic solvent(s);
iv) mixing the enteric coated pellets with at least one tablet excipient selected from filler, binder, disintegrant, lubricating agent, sweetener and flavor;
v) compressing the blend of step (iv) into tablets.

The compressed tablet when analyzed in-vitro releases
i) not more than 15% active ingredient in acidic pH at least for 1 to 2 hour and
ii) not less than 70% of active ingredient in near neutral to alkaline media within 1 hour.

The invention also provides orodispersible tablet compositions that disintegrate rapidly in the oral cavity and a process for the preparation of such composition.

The active ingredient used in the present invention is selected from the group of omeprazole, pantoprazole, lansoprazole, rabeprazole, duloxetine or their pharmaceutically acceptable salts such as rabeprazole sodium or their enantiomers such as esomeprazole or pharmaceutically acceptable salts of their enantiomers such as esomeprazole magnesium trihydrate or mixtures thereof.

### DETAILED DESCRIPTION OF INVENTION

The present invention provides a multiple unit tablet compositions comprising of enteric coated pellets and at least one tablet excipient, wherein each pellet comprises:
i) a core comprising active ingredient(s);
ii) optionally a separating layer coated on the core;
iii) at least two enteric layers comprising of enteric polymers and plasticizer either coated on the core or on the separating layer to obtain enteric coated pellets, such that the last enteric layer is formed from a solution comprising of enteric polymer and plasticizer in organic solvent(s); the total enteric polymers being at least 20% by weight of the enteric coated pellets and plasticizer up to 15% by weight of enteric polymers, resulting in no appreciable change in release profile of active ingredient on compression of enteric coated pellets into tablets.

The present invention also provides a process for the preparation of multiple unit compositions comprising of enteric coated pellets, exhibiting no appreciable change in release profile of active ingredient on compression, comprising steps:
i) preparation of a core comprising active ingredient(s);
ii) optionally creating a separating layer on the core;
iii) coating core or separating layer coated core with at least two enteric layers comprising of enteric polymers and plasticizer to obtain enteric coated pellets, wherein the enteric polymers is at least 20% by weight of the enteric coated pellets and plasticizer is up to 15% by weight of enteric polymers, wherein last enteric layer is formed from a solution comprising of enteric polymer(s) and plasticizer in organic solvent(s);
iv) mixing the enteric coated pellets with at least one tablet excipient selected from filler, binder, disintegrant, lubricating agent, sweetener and flavor;
v) compressing the blend of step (iv) into tablets.

All expression of percentage, ratio, proportions and the like stated herein are in weight units unless otherwise stated. The term “enteric coated pellets” refer to pellets that are coated with last enteric layer.

The various stages of the multiple unit tablet composition comprising of enteric coated pellets and the process for the preparation of these compositions are as follows:

### Stage 1: Preparation of Core:

A core for the preparation of enteric coated pellets is prepared by applying layer comprising active ingredient on inert seeds. Such inert seeds are conventionally used in pharmaceutical industry and are generally made of sugar and starch. However, other ingredients such as microcrystalline cellulose (MCC), carbohydrates, cellulose, resins, wax, different oxides and other materials may also be used for the preparation of inert seeds. The seeds are of about 100 to 710 microns, preferably about 150 to 600 microns, more preferably about 200 to 450 microns. The inert seed is generally about 7.5 to 60% by weight of the enteric coated pellets.

The process involves deposition of layer comprising active ingredient, binder and optionally other pharmaceutically acceptable ingredients on the inert seeds. The application of layer comprising active ingredient can be done simultaneous with binder or alternating with binder.

When suspension layering method is employed, the active ingredient and binder are dispersed and/or dissolved in a suitable solvent to which other pharmaceutical ingredient(s) is added. The resulting dispersion is sprayed on inert seeds to obtain core comprising active ingredient.

When powder layering method is used, the binder solution or dispersion in a suitable solvent is sprayed on inert seeds and powder blend comprising active ingredient and other pharmaceutical ingredient is layered on the wetted inert seeds. This alternating process of wetting inert seeds with binder and layering powder blend comprising active ingredient is continued till the entire powder blend is used up to obtain core comprising active ingredient.

Alternatively, the core may comprise of matrix monolithic system, wherein active ingredient, binder and other pharmaceutical ingredients are mixed and granulated using a suitable solvent to obtain granules. These granules are extruded and spheronized to obtain core comprising active ingredient.

The active ingredients used in the present invention is selected from omeprazole, pantoprazole, lansoprazole, rabeprazole, substituted phenylmethylsulfinyl-1H-benzimidazoles, cycloheptapyridin-9-ylsulfinyl-1H-benzimidazoles or pyridin-2-ylmethyl sulfinylthieno-imidazoles, leminoprazole, 2-(4-methoxy-6,7,8,9-tetrahydro-5H-cyclo hepta[b]pyridin-9-ylsulfinyl)-1H-benzimidazole (nepaprazole) and duloxetine or their pharmaceutically acceptable salts such as rabeprazole sodium or their enantiomers such as esomeprazole or pharmaceutically acceptable salts of their enantiomers such as esomeprazole magnesium and mixtures thereof. The hydrated forms of the active ingredient for example esomeprazole magnesium trihydrate, pantoprazole sodium sesquihydrate, etc. are also included in the scope of the invention.

For the purpose of illustrating the invention, rabeprazole sodium is used as an active ingredient.

When a powder layering or suspension layering method is used, the median particle size of active ingredient is below 100 microns, preferably below 75 microns and more preferably below 50 microns.

The amount of the active ingredient in enteric coated pellets is below 50%, more preferably below 30% and most preferably below 20% by weight of enteric coated pellets.

The active ingredient is mixed with binder and/or other pharmaceutical ingredients to prepare the core.

Binder is selected from the group of cellulose derivatives such as hydroxypropylmethylcellulose (HPMC), hydroxypropylcellulose (HPC), ethyl cellulose, carboxymethylcellulose sodium, methylcellulose, hydroxyethylcellulose, microcrystalline cellulose; polymethacrylates, sugars such as lactose, sucrose etc.; polyvinylpyrrolidone (PVP), waxes, fatty alcohols such as stearyl alcohol, cetyl alcohol; gelatin, starch, pregelatinized starch, carbomer; gums like xanthan gum, guar gum, acacia, alginates and mixtures thereof. Polymethacrylates such as Eudragit RL30D, Eudragit RLPO, Eudragit RL, Eudragit RS30D, Eudragit RSPO, Eudragit RS, Eudragit NE30D, Eudragit NE40D, Eudragit NM30D and Eudragit E are used. The binder is preferably selected from HPMC, HPC, PVP, microcrystalline cellulose, lactose and mixtures thereof.

Binder present in the core is up to about 40% by weight of enteric coated pellets, preferably from about 0.01% to about 20% by weight of enteric coated pellets, more preferably from about 0.1% to about 10% by weight and most preferably from 0.5% to about 5% by weight of enteric coated pellets.

Other pharmaceutical ingredients used in the preparation of the core is selected from one or more fillers, anti-adherents, surfactants, buffers, alkaline substances, disintegrating agents, pigments, colours and mixtures thereof.

Fillers are selected from the group of carbohydrates such as glucose, lactose, mannitol, sucrose, dextrose, sorbitol, fructose, sorbitol, compressible sugar, etc; calcium phosphate, dibasic calcium phosphate, tribasic calcium phosphate, starch, pregelatinized starch, starch 1500, cyclodextrins and its derivatives; carboxymethylcellulose and its salts such as sodium, potassium and calcium salt; calcium sulfate, microcrystalline cellulose, cetyl alcohol, stearyl alcohol, waxes and mixtures thereof.

Surfactants are selected from the group of cationic surfactant, non-ionic surfactant and anionic surfactant and is preferably selected from sodium lauryl sulfate, polysorbates, sorbitan esters, poloxamers, fatty acid esters and ethers of polyethylene glycol, alkyl phenoxy polyethylene glycols, block polymers of polyethylene and polypropylene oxides, oleic acid and its salt, bile salts and their conjugates, octoxynol, polyoxyethylene and its derivatives such as castor oil derivatives polyoxyethylene monoalkyl ethers, sucrose esters, lanolin esters and ethers, lauric acid and its salts, alkyl sulfate and its salts, fatty acid and its salts and mixtures thereof.

Anti-adherents are selected from talc, colloidal silicon dioxide, magnesium stearate, calcium stearate, glyceryl monostearate, glyceryl behenate, sodium lauryl sulfate, stearic acid and mixtures thereof.

Buffers and alkaline substances may be used singly or in mixtures and are selected from the group of alkali and alkaline earth metals hydroxides, carbonate, bicarbonate, sulphate, phosphates and oxides; and amino acids. It is preferably selected from one or more of oxides, hydroxides, carbonates, bicarbonates, phosphates and sulphates of sodium, potassium, calcium, zinc, magnesium and aluminium; the composite aluminium/magnesium compounds Al₂O₃.6MgO.CO₂.12H₂O or MgO.Al₂O₃.2SiO₂.nH₂O, where n is not an integer but less than 2.

Buffers such as acetate, phosphate, borate, bicarbonate, carbonate, succinate, tris buffer, organic acid buffer and mixtures thereof may also be used. Preferably alkaline substance from monobasic sodium phosphate, dibasic sodium phosphate, tribasic sodium phosphate, sodium hydroxide, potassium hydroxide, sodium lauryl sulphate, magnesium carbonate, calcium carbonate, magnesium oxide and mixtures thereof are used.

Disintegrating agent is selected from the group of sodium starch glycolate, crospovidone, cross linked carboxymethylcellulose and its salts such as sodium, potassium and calcium salt; starch, modified starch, pregelatinized starch, starch 1500, microcrystalline cellulose and mixtures thereof.

Pigments and colours are selected from pharmaceutically acceptable pigments and colours. Titanium oxide, iron oxide colours such as iron oxide red; lake colours such as lake of sunset yellow and mixtures thereof are preferably used.

The solvent is selected from aqueous, alcoholic, hydro-alcoholic and organic solvents and is preferably selected from water, methanol, ethanol, isopropanol, acetone, dichloromethane and mixtures thereof. The solvent of choice for the preparation of the core is water.

The prepared core is dried to moisture content of less than 5%, preferably less than 3% and more preferably less than 2% by weight of cores.

### Stage II: Formation of Separating Layer:

The core is optionally coated with a separating layer comprising of binder and optionally other pharmaceutical ingredients. The binder is either dispersed or dissolved in a solvent and the other pharmaceutically ingredients are added. The resulting solution or dispersion is sprayed on the core to form the separating layer coated core. The separating layered coated cores are dried to moisture content of less than 5%, preferably less than 3% and more preferably less than 2% by weight of separating layer coated cores.

The other pharmaceutical ingredients used in the separating layer are same as those present in core.

Binder in the separating layer is up to 15%, more preferably from 0.5 to 10% and most preferably from 1.5 to 5% by weight of enteric coated pellets.

### Stage III: Preparation of Enteric Coated Pellets:

The cores or separating layer coated cores of the present invention are coated with at least two enteric layers comprising of enteric polymers and plasticizer such that the last enteric layer is formed from a solution comprising of enteric polymer and plasticizer in organic solvent(s).

The process of coating of last enteric layer comprises steps of:
a) dissolving enteric polymer(s) in organic solvent selected from methanol, ethanol, isopropanol, dichloromethane, acetone and mixtures thereof;
b) adding plasticizer to the polymer(s) solution of step (a);
c) spraying the solution of step (b) on the preceding enteric layer in fluid bed bottom spray processor to obtain enteric coated pellets.

The enteric layers are formed from a solution or dispersion comprising of enteric polymers and plasticizers in organic solvent(s) or water but the last enteric layer is formed from a solution comprising of enteric polymer and plasticizer in organic solvent(s). The organic solvents used for the formation of the last layer may contain water.

Enteric polymer in the enteric layers is selected from methacrylic acid copolymers, cellulosic polymers, polyvinyl alcohol phthalate, polyvinyl acetate phthalate, shellac and mixtures thereof.

Methacrylic acid copolymers is selected from Eudragit L30D55 (Type C), Eudragit L10055 (Type C), Eudragit L100 (Type A), Eudragit L12.5, (Type A), Eudragit S100 (Type B), Eudragit S12.5 (Type B) and Eudragit FS30D.

Cellulosic polymer is selected from cellulose acetate phthalate (CAP), cellulose acetate trimelliate (CAT), hydroxypropylmethylcellulose phthalate (HPMCP), cellulose propionate phthalate, hydroxypropylmethylcellulose acetate succinate (HPMCAS), cellulose acetate maleate and hydroxypropylmethylcellulose hexahydrophthalate.

The total enteric polymers are at least 20%, preferably 30% to 70%, more preferably 40% to 60% by weight of the enteric coated pellets.

The enteric coated pellets comprises of two or more enteric layers, which differ in composition and ratio.

In one aspect of the invention, the enteric coated pellets have two enteric layers, the ratio of enteric polymer in these two layers is 0.8:0.2 to 0.2:0.8. Enteric polymer or polymers is preferred in the range of 0.7: to 0.3 to 0.3:0.7, more preferably from 0.6:0.4 to 0.4:0.6.

In another aspect of the invention, the enteric coated pellets have three or more enteric layers, the enteric polymer(s) in one layer is at least 10% by weight of the total enteric polymers.

The organic solvent used is selected from methanol, ethanol, isopropanol, acetone, dichloromethane and mixtures thereof.

Plasticizer is selected from the group of hydrophilic and/or hydrophobic plasticizers and is selected from polyethylene glycol, triacetin, triethylcitrate, acetyl triethylcitrate, miglyol, cetyl alcohol, acetyltributylcitrate, diethyl phthalate, dibutyl phthalate, propylene glycol, hydrogenated oils, dibutylsebacate, meglumine and mixtures thereof and is preferably dibutyl sebacate. Plasticizers in the enteric layers is up to 15%, preferably up to 12.5%, more preferably up to 10% by weight of enteric polymer.

Enteric layer optionally comprises of anti-adherents, pigments, colorants, surfactants and anti-foaming agents such as silicone oil.

Anti-adherent in the enteric layers is selected from talc, colloidal silicon dioxide, glyceryl monostearate, stearic acid, kaolin, magnesium stearate, calcium stearate, sodium stearyl fumarate, glyceryl behenate, starch and mixtures thereof. These are present up to 30%, preferably 1% to 20%, more preferably 5% to 15% and most preferably about 10% by weight of the enteric coated pellets.

Pigments and colours are selected from pharmaceutically acceptable pigments and colours such as titanium oxide, iron oxide red and lake of sunset yellow.

Surfactant is selected from sodium lauryl sulfate and polysorbates such as polysorbate 80 and mixtures thereof.

These enteric coated pellets are analyzed in-vitro in:
i) 0.1 N HCl or pH 1.2 buffer for 1 to 2 hour, and
ii) Near neutral to alkaline buffer with or without surfactant for 1 hour.

The general release specification for enteric coated pellets comprising active ingredients is as follows.

| Time | | Media | Specification |
|---|---|---|---|
| 1-2 | hours | 0.1 N HCl or pH 1.2 buffer | Not more than 15% to be |
| | | | released |
| 1 | hour | Near neutral to alkaline | Not less than 70% to be released |
| | | buffer with or without | |
| | | surfactant | |

Stage IV: Compression into Tablets:

Enteric coated pellets prepared above are compressed into swallowable tablets or orally dispersible tablets. The size of enteric coated pellets for compression should be less than 850 microns, preferably about 250 to 710 microns, more preferably about 300 to 600 microns and most preferably about 425 to 600 microns.

The tablets comprise at least one tablet excipient selected from filler, binder, disintegrant, lubricants, sweetener, flavor and color.

Fillers and binders used in the preparation of tablet include those used in the preparation of core.

Disintegrant is selected from crospovidone, croscarmellose sodium, sodium starch glycolate, croscarmellose calcium, hydroxypropylcellulose, starch, pregelatinized starch, modified starch, starch 1500, microcrystalline cellulose, sodium carboxymethylcellulose, sodium bicarbonate, potassium bicarbonate, calcium carbonate, ammonium bicarbonate, malic acid, citric acid, tartaric acid and mixtures thereof.

Disintegrant is up to 75%, preferably from 1to 50%, more preferably from 2.5 to 25% and most preferably from 5 to 15% by weight of the tablet.

Lubricants are selected from talc, colloidal silicon dioxide, magnesium stearate, glyceryl monostearate, glyceryl behenate, stearic acid, sodium stearyl fumarate, calcium stearate and sodium benzoate.

Sweeteners include saccharine sodium, calcium saccharin, cyclamate sodium, acesulfame potassium, erythritol, xylitol, steviosides, aspartame, sorbitol, sucralose glycyrrhizin and its derivatives and thaumatin. Glycyrrhizin and its derivatives include monoammonium glycyrrhizinate, dipotassium glycyrrhizinate, monopotassium glycyrrhizinate, licorice extract powder, licorice spray extract powder, glycyrrhizic acid powder, monosodium glycyrrhizinate, glycyrrhiza flavone, and disodium glycyrrhizinate.

Any suitable natural, semi-synthetic and synthetic flavours and colours may be used.

The shape and dimension of the tablets has no impact but circular tablets of 5 mm to 20 mm are preferred.

The compressed tablets are either a swallowable tablet or orally dispersible tablet and have hardness from 1 kp to 20 kp.

In case of orally dispersible tablets, the disintegrating time in the oral cavity is not more than 3 minutes, preferably not more than 2 minutes more preferably not more than 1 minute and most preferably not more than 0.5 minute.

The compressed tablets are analyzed in-vitro under similar set of conditions as that of enteric coated pellets. The release specification of these compressed tablets is same as that of enteric coated pellets.

The invention is now described with non-limiting examples for the preparation of tablet dosage form comprising enteric coated pellets.

### EXAMPLE 1

### Multiple Unit Tablet Composition With Two Enteric Favers:

Core

| | | |
|---|---|---|
| Rabeprazole Sodium | 20 | mg |
| MCC pellets (40-60# ASTM) | 30 | mg |
| HPMC E15 | 2 | mg |
| Sodium Hydroxide | 2 | mg |
| Sodium Starch glycolate | 0.4 | mg |
| Talc | 2 | mg |
| Methanol | 42.24 | mg |
| Purified water | 63.36 | mg |

Separating Layer

| | | |
|---|---|---|
| HPMC E15 | 3.95 | mg |
| Ethyl cellulose | 1.69 | mg |
| Methylene chloride | 64.3 | mg |
| Methanol | 42.86 | mg |

Enteric layer 1

| | | |
|---|---|---|
| Eudragit L30D 55 (solid content) | 46.53 | mg |
| Dibutylsebacate | 6.2 | mg |
| Glyceryl monostearate | 9.31 | mg |
| Polysorbate 80 | 1.55 | mg |
| Talc | 2.33 | mg |
| Iron oxide red | 1.16 | mg |
| Purified water | 159.75 | mg |

Enteric layer 2

| | | |
|---|---|---|
| HPMCP HP55 | 32.28 | mg |
| Dibutylsebacate | 3.23 | mg |
| Methylene chloride | 337.33 | mg |
| Methanol | 337.33 | mg |

Tablet

| | | |
|---|---|---|
| Mannitol SD 200 | 465.87 | mg |
| Crospovidone | 75 | mg |
| Magnesium stearate | 7.5 | mg |
| Colloidal silicone dioxide | 11.25 | mg |
| Aspartame | 18.25 | mg |
| Strawberry flavour | 7.5 | mg |

### Core:

a) HPMC E15 was dispersed and dissolved in the mixture of methanol and water to obtain binder solution.
b) A solution of sodium hydroxide in water was added to the binder solution of step (a).
c) Rabeprazole sodium, sodium starch glycolate and talc were added to the solution obtained in step (b) to obtain drug dispersion.
d) The drug dispersion was sprayed on MCC pellets in fluid bed bottom spray processor and dried in the same equipment to obtain core.

### Separating Layer:

a) Ethyl cellulose and HPMC E15 were dispersed and dissolved in the mixture of methanol and methylene chloride.
b) The resulting solution was sprayed on cores in fluid bed bottom spray processor followed by drying to obtain separating layer coated cores.

### Enteric Layer 1:

a) A solution of polysorbate 80 in water was prepared and was heated to about 70° C.
b) The above surfactant solution was added under high speed stirring to the hot mixture of glyceryl monostearate and dibutyl sebacate followed by addition of water.
c) The dispersion of step (b) was added to Eudragit L30D 55 dispersion followed by addition of dispersion of talc and Iron oxide red.
d) The resulting dispersion was homogenized and filtered.
e) The resulting dispersion was sprayed on separating layer coated cores in fluid bed bottom spray processor followed by drying to obtain first enteric layer coated cores.

### Enteric Layer 2:

a) HPMCP HP 55 was dispersed and dissolved in the mixture of methanol and methylene chloride.
b) Dibutyl sebacate was added to the above solution.
c) The resulting solution was sprayed on first enteric layer coated cores in fluid bed bottom spray processor followed by drying to obtain enteric coated pellets.
   Compression into Tablets:
a) Enteric coated pellets (30-40#) were mixed with mannitol SD 200, crospovidone, aspartame and strawberry flavour in octagonal blender.
b) Magnesium stearate and colloidal silicon dioxide were blended with mixture of step (a) in octagonal blender.
c) This blend was compressed into orally dispersible tablets corresponding to 20 mg of rabeprazole sodium using single rotary compression machine.

### EXAMPLE 2

### Multiple Unit Tablet Composition With One Enteric Layer:

Enteric layer 1

| | | |
|---|---|---|
| Eudragit L30D 55 (solid content) | 78.79 | mg |
| Dibutylsebacate | 11.26 | mg |
| Glyceryl monostearate | 16.88 | mg |
| Polysorbate 80 | 2.81 | mg |
| Talc | 3.94 | mg |
| Iron oxide red | 1.97 | mg |
| Purified water | 278.79 | mg |

Tablet

| | | |
|---|---|---|
| Mannitol SD 200 | 490.31 | mg |
| Crospovidone | 80 | mg |
| Magnesium stearate | 12 | mg |
| Colloidal silicone dioxide | 16 | mg |
| Aspartame | 16 | mg |
| Strawberry flavour | 8 | mg |

The separating layer coated core as prepared in example 1 is coated with only one enteric layer from aqueous media.

### Enteric Layer:

Enteric coated pellets were prepared by coating separating layer coated cores with one enteric layer as described in enteric layer 1 of example 1.

Compression into Tablets:
a) The preparation of blend is same as described in example 1.
b) This blend was compressed into orally dispersible tablets corresponding to 20 mg of rabeprazole sodium using single rotary compression machine.

### EXAMPLE 3

### Multiple Unit Tablet Composition With One Enteric Layer:

The separating layer coated core as prepared in example 1 is coated with only one enteric layer from organic solvents.

Enteric layer 1

| | |
|---|---|
| HPMCP HP55 | 78.79 mg |
| Dibutylsebacate | 7.88 mg |
| Methylene chloride | 823.37 mg |
| Methanol | 823.37 mg |

### Enteric Layer:

Enteric coated pellets were prepared by coating separating layer coated cores with one enteric layer as described in enteric layer 2 of example 1.

### EXAMPLE 4

### Multiple Unit Tablet Composition With Two Enteric Layers:

Core

| | | |
|---|---|---|
| Rabeprazole Sodium | 20 | mg |
| MCC pellets (40-60# ASTM) | 30 | mg |
| HPMC E15 | 2 | mg |
| Sodium Hydroxide | 2 | mg |
| Talc | 2 | mg |
| Methanol | 42.24 | mg |
| Purified water | 63.36 | mg |

Separating Layer

| | | |
|---|---|---|
| HPMC E15 | 14 | mg |
| Light magnesium oxide | 2.8 | mg |
| Methylene chloride | 191.52 | mg |
| Methanol | 127.68 | mg |

Enteric layer 1

| | | |
|---|---|---|
| Eudragit L30D 55 (solid content) | 54.6 | mg |
| Dibutylsebacate | 3.11 | mg |
| Glyceryl monostearate | 2.08 | mg |
| Polysorbate 80 | 0.52 | mg |
| Talc | 2.73 | mg |
| Iron oxide red | 0.55 | mg |
| Purified water | 125.39 | mg |

Enteric layer 2

| | | |
|---|---|---|
| HPMCP HP55 | 58.24 | mg |
| Dibytylsebacate | 5.82 | mg |
| Methylene chloride | 608.57 | mg |
| Methanol | 608.57 | mg |

Tablet

| | | |
|---|---|---|
| Mannitol SD 200 | 534.55 | mg |
| Crospovidone | 100 | mg |
| Microcrystalline cellulose pH 102 | 50 | mg |
| Hydroxypropylcellulose LH 31 | 50 | mg |
| Magnesium stearate | 25 | mg |
| Colloidal silicone dioxide | 20 | mg |
| Aspartame | 10 | mg |
| Strawberry flavour | 10 | mg |

### Core:

a) The core comprising rabeprazole sodium was prepared as described in example 1.

### Separating Layer:

a) HPMC E15 was dispersed and dissolved in the mixture of methanol and methylene chloride.
b) Light magnesium oxide was added to the above solution and the resulting dispersion was filtered.
c) The filtered dispersion was sprayed on cores in fluid bed bottom spray processor followed by drying to obtain separating layer coated cores.

### Enteric Layers:

a) Enteric coated pellets were prepared by coating separating layer coated cores with first enteric layer followed by second enteric layer as described in example 1.
   Compression into Tablets:
a) Enteric coated pellets (30-40#) were mixed with mannitol SD 200 in octagonal blender for 15 minutes.
b) Microcrystalline cellulose pH 102, crospovidone, hydroxypropylcellulose LH 31, aspartame and strawberry flavour were added to the mixture of step (a) and were blended in octagonal blender.
c) Magnesium stearate and colloidal silicon dioxide were blended with step (b) mixture in octagonal blender.
d) This blend was compressed into orally dispersible tablets corresponding to 20 mg of rabeprazole sodium using single rotary compression machine.

### EXAMPLE 5

### Multiple Unit Tablet Composition With Three Enteric Layers:

Core

| | |
|---|---|
| Rabeprazole Sodium | 20 |
| MCC pellets (40-60# ASTM) | 30 |
| HPMC E15 | 2 |
| Sodium Hydroxide | 2 |
| Talc | 2 |
| Methanol | 41.6 |
| Purified water | 62.4 |

Separating Layer

| | |
|---|---|
| HPMC E15 | 0.34 |
| Ethyl cellulose | 0.78 |
| Methylene chloride | 10.64 |
| Methanol | 10.64 |

Enteric layer 1

| | |
|---|---|
| Eudragit L10055 | 10 |
| Dibutylsebacate | 1.43 |
| Glyceryl monostearate | 2.85 |
| Methylene chloride | 135.66 |
| Methanol | 135.66 |

Enteric layer 2

| | |
|---|---|
| Eudragit L30D 55 (solid content) | 32.13 |
| Dibutylsebacate | 4.28 |
| Glyceryl monostearate | 6.43 |
| Polysorbate 80 | 1.07 |
| Talc | 1.61 |
| Iron oxide red | 1.61 |
| Purified water | 113.55 |

Enteric layer 3

| | |
|---|---|
| HPMCP HP55 | 11.42 |
| Dibutylsebacate | 1.14 |
| Methylene chloride | 119.32 |
| Methanol | 119.32 |

Tablet

| | |
|---|---|
| Mannitol SD 200 | 518.16 |
| Crospovidone | 37.5 |
| Sodium starch glycolate | 18.75 |
| Magnesium stearate | 7.5 |
| Colloidal silicone dioxide | 11.25 |
| Aspartame | 18.25 |
| Strawberry flavour | 7.5 |

The core and separated layer coated cores were prepared as described in example 1.

Enteric layers:
a) Eudragit L10055 was dispersed and dissolved in the mixture of methanol and methylene chloride.
b) Glyceryl monostearate and dibutyl sebacate was added to the above solution.
c) The resulting solution was sprayed on separated layer coated cores in fluid bed bottom spray processor followed by drying to obtain first enteric layer coated cores.

The process of coating enteric layer 2 and enteric layer 3 is same as described in enteric layer 1 and enteric layer 2 respectively of example 1.

Compression into Tablets:
a) The preparation of blend is same as described in example 1.
b) This blend was compressed into orally dispersible tablets corresponding to 20 mg of rabeprazole sodium using single rotary compression machine.

### EXAMPLE 6

### Multiple Unit Tablet Composition With Three Enteric Layers:

Core

| | |
|---|---|
| Rabeprazole sodium | 20 |
| MCC pellets 50-60# ASTM | 30 |
| HPMC E15 | 2 |
| Sodium Hydroxide | 2 |
| Talc | 2 |
| Methanol | 42.24 |
| Purified water | 63.36 |

Separating Layer

| | |
|---|---|
| HPMC E15 | 14 |
| Light magnesium oxide | 2.8 |
| Methylene chloride | 191.52 |
| Methanol | 127.68 |

Enteric layer 1

| | |
|---|---|
| Eudragit L30D55 (solid content) | 54.6 |
| Dibutylsebacate | 3.11 |
| Glyceryl monostearate | 2.08 |
| Polysorbate 80 | 0.52 |
| Talc | 2.73 |
| Iron oxide red | 0.55 |
| Purified water | 125.39 |

Enteric layer 2

| | |
|---|---|
| HPMCP HP 55 | 43.68 |
| Dibutylsebacate | 4.39 |
| Methylene Chloride | 456.48 |
| Methanol | 456.48 |

Enteric layer 3

| | |
|---|---|
| Eudragit L 100 | 10.19 |
| Eudragit S100 | 4.37 |
| Dibutylsebacate | 1.46 |
| Isopropanol | 304.38 |

Tablet

| | |
|---|---|
| Mannitol SD 200 | 534.52 |
| Crospovidone | 100 |
| Microcrystalline cellulose PH102 | 50 |
| Hydroxypropylcellulose LH31 | 50 |
| Aspartame | 10 |
| Strawberry | 10 |
| Magnesium stearate | 25 |
| Colloidal silicon dioxide | 20 |

The preparation of core comprising rabeprazole sodium and separating layer coated cores is same as described in example 2.

The process of coating of enteric layer 1 and enteric layer 2 is same as described in enteric layer 1 and enteric layer 2 respectively of example 1.

Enteric layer 3:
a) Eudragit L100 and Eudragit S100 was dispersed and dissolved in isopropanol.
b) Dibutylsebacate was added to the above solution.
c) The resulting solution was sprayed on second enteric layer coated cores in fluid bed bottom spray processor followed by drying to obtain enteric coated pellets.
   Compression into Tablets:
a) The preparation of blend is same as described in example 2.
b) This blend was compressed into orally dispersible tablets corresponding to 20 mg of rabeprazole sodium using single rotary compression machine.

### EXAMPLE 7

### Multiple Unit Tablet Composition With Two Enteric Layers:

Core

| | |
|---|---|
| Lansoprazole | 30 |
| Lactose | 30 |
| Trisodium orthophosphate | 6 |
| Sodium lauryl sulphate | 3 |
| Povidone K30 | 1.2 |
| Polyethylene glycol 6000 | 1.2 |
| Purified water | 8.57 |

Enteric layer 1

| | |
|---|---|
| Eudragit L30D55 | 16.06 |
| Dibutylsebacate | 2.14 |
| Glyceryl monostearate | 3.21 |
| Polysorbate 80 | 0.54 |
| Talc | 0.8 |
| Titanium dioxide | 0.4 |
| Purified water | 55.16 |

Enteric layer 2

| | |
|---|---|
| HPMCP HP 55 | 21.42 |
| Dibutylsebacate | 2.14 |
| Purified water | 29.85 |
| Methylene Chloride | 223.85 |
| Methanol | 194.01 |

Tablet

| | |
|---|---|
| Mannitol SD 200 | 362 |
| Crospovidone | 57.5 |
| Aspartame | 11.5 |
| Strawberry flavour | 5.75 |
| Magnesium stearate | 8.64 |
| Colloidal silicon dioxide | 11.5 |

### Core:

a) Lansoprazole, lactose, trisodium orthophosphate and sodium lauryl sulphate were blended in planetary mixer for about 10 minutes.
b) The drug mixture was granulated using aqueous solution of PVP K30 and polyethylene glycol 6000 to obtain wet mass.
c) The wet mass was extruded using a screw type extruder to obtain extrudates.
d) The extrudates were converted into spherical form in a spheronizer.
e) The particles were dried and sized to obtain cores (25-45# ASTM).

### Enteric Layers:

Dispersion of enteric layer 1 and solution of enteric layer 2 are prepared as described in example 1 and the core comprising lansoprazole is coated with first enteric layer followed by second enteric layer to obtain enteric coated pellets.

Compression into Tablets:
a) Enteric coated pellets were mixed with mannitol SD 200, crospovidone, aspartame and strawberry flavour in octagonal blender for 15 minutes.
b) Magnesium stearate and colloidal silicon dioxide were blended with step a) mixture in octagonal blender.
c) This blend was compressed into orally dispersible tablets corresponding to 20 mg of rabeprazole sodium using single rotary compression machine.

The results from the tests on acid release and release in near neutral to alkaline pH (buffer release) of the enteric coated pellets and compressed tablets are disclosed in Table I below.

TABLE I

| Example | Acid release, | Acid release, | Buffer release, | Buffer release, |
|---|---|---|---|---|
| No. | pellets (%) | tablets (%) | pellets (%) | tablets (%) |
| 1 | 1.44 | 3.63 | 92.7 | 95.78 |
| 2 | 7.64 | 42.67 | 91.94 | — |
| 3 | 26.11 | — | — | — |
| 4 | 4.47 | 0.0 | 85.7 | 93.1 |
| 5 | 1.71 | 6.96 | 96.08 | 90.58 |
| 6 | 4.12 | 1.98 | 87 | 87.2 |
| 7 | 4.87 | 5.36 | 95.03 | 86.5 |

Example 2 illustrate that when separating layer coated cores are coated with one enteric layer from aqueous medium, the enteric coated pellets exhibit acceptable release profile in acidic medium. However, when these enteric coated pellets are compressed into tablets, it fails to protect the acid labile drug when analyzed in acidic medium.

Example 3 illustrate that when separating layer coated cores are coated with one enteric layer from organic solvent, the enteric coated pellets fails to protect the acid labile drug when analyzed in acidic medium.

Examples 1 and 4-7 illustrate that separating layer coated cores should be coated with at least 2 enteric layers, wherein the last enteric layer should be formed using organic solvent such that there is there is no appreciable change in the release profile of active ingredient before and after compression, and the acid labile drug is protected when analyzed in acidic medium.

It should be noted that the multiple enteric layers are essential to prepare multiple unit tablet compositions comprising of enteric coated pellets such that there is no appreciable change in the release profile of active ingredient before and after compression, and the acid labile drug is protected when analyzed in acidic medium.

1. A multiple unit composition comprising enteric coated pellets and at least one tablet excipient, wherein each pellet comprises:
i) a core comprising active ingredient(s);
ii) optionally a separating layer coated on the core; and
iii) at least two enteric layers comprising of enteric polymers and plasticizer either coated on the core or on the separating layer to obtain enteric coated pellets, such that the last enteric layer is formed from a solution comprising of enteric polymer and plasticizer in organic solvent(s); the total enteric polymers being at least 20% by weight of the enteric coated pellets and plasticizer up to 15% by weight of enteric polymers, resulting in no appreciable change in release profile of active ingredient on compression of enteric coated pellets into tablets.

2. The multiple unit composition of claim 1, wherein the total enteric polymers are 30% to 70% by weight of enteric coated pellets, preferably 40% to 60% by weight of enteric coated pellets.

3. The multiple unit composition of claim 1, having two enteric layers, wherein the ratio of enteric polymer present in the two layers is 0.8:0.2 to 0.2:0.8.

4. The multiple unit composition of claim 1, having three or more enteric layers, wherein the enteric polymer(s) in one layer is at least 10% by weight of the total enteric polymers.

5. The multiple unit composition of claim 1, wherein the enteric polymers are selected from the group consisting of methacrylic acid copolymer Type A, methacrylic acid copolymer Type B, methacrylic acid copolymer Type C, hydroxypropylmethylcellulose phthalate, hydroxypropylmethylcellulose acetate succinate and mixtures thereof.

6. The multiple unit composition of claim 1, wherein the plasticizer is up to 12.5% by weight of enteric polymers, preferably up to 10% by weight of enteric polymers.

7. The multiple unit composition of claim 1, wherein the plasticizer is selected from the group consisting of triacetin, triethylcitrate, acetyltriethyl citrate, acetyltributyl citrate, dibutyl phthalate, dibutyl sebacate, diethyl phthalate, polyethylene glycol, hydrogenated oil, cetyl alcohol, miglyol, meglumine, propylene glycol and mixtures thereof and is preferably dibutyl sebacate.

8. The multiple unit composition of claim 1, wherein the organic solvent is selected from the group consisting of methanol, ethanol, isopropanol, dichloromethane, acetone and mixtures thereof.

9. The multiple unit composition of claim 1, wherein the active ingredient is selected from the group consisting of omeprazole, pantoprazole, lansoprazole, rabeprazole, duloxetine or their pharmaceutically acceptable salts such as rabeprazole sodium or their enantiomers such as esomeprazole or pharmaceutically acceptable salts of their enantiomers such as esomeprazole magnesium trihydrate and mixtures thereof.

10. The multiple unit composition of claim 1, wherein the tablet excipient is selected from the group consisting of filler, binder, disintegrant, lubricating agent, sweetener, flavor and mixtures thereof.

11. The multiple unit composition of claim 1, wherein the tablet is orally dispersible tablet.

12. A process for the preparation of multiple unit compositions comprising enteric coated pellets, exhibiting no appreciable change in release profile of active ingredient on compression, comprising steps:
i) preparation of a core comprising active ingredient(s);
ii) optionally creating a separating layer on the core;
iii) coating core or separating layer coated core with at least two enteric layers comprising of enteric polymers and plasticizer to obtain enteric coated pellets, wherein the enteric polymers is at least 20% by weight of the enteric coated pellets and plasticizer is up to 15% by weight of enteric polymers, wherein last enteric layer is formed from a solution comprising of enteric polymer(s) and plasticizer in organic solvent(s);
iv) mixing the enteric coated pellets with at least one tablet excipient selected from the group consisting of filler, binder, disintegrant, lubricating agent, sweetener and flavor; and
v) compressing the blend of step (iv) into tablets.

13. The process of claim 12, wherein the coating with last enteric layer comprises the steps of:
a) dissolving enteric polymer(s) in organic solvent selected from the group consisting of methanol, ethanol, isopropanol, dichloromethane, acetone and mixtures thereof;
b) adding plasticizer to the enteric polymer(s) solution of step (a); and
c) spraying the solution of step (b) on the preceding enteric layer in fluid bed bottom spray processor to obtain enteric coated pellets.

14. The process of claim 12, wherein the total enteric polymers are 30% to 70%, preferably 40% to 60% by weight of enteric coated pellets.

15. The process of claim 12, wherein the enteric polymers are selected from the group consisting of methacrylic acid copolymer Type A1 methacrylic acid copolymer Type B, methacrylic acid copolymer Type C, hydroxypropylmethylcellulose phthalate, hydroxypropylmethylcellulose acetate succinate and their suitable mixtures.

16. The process of claim 12, wherein the plasticizer is up to 12.5% by weight of enteric polymers, preferably up to 10% by weight of enteric polymers.

17. The process of claim 12, wherein the plasticizer is selected from the group consisting of triacetin, triethylcitrate, acetyltriethyl citrate, acetyltributyl citrate, dibutyl phthalate, dibutyl sebacate, diethyl phthalate, polyethylene glycol, hydrogenated oil, cetyl alcohol, miglyol, meglumine, propylene glycol and mixtures thereof and is preferably dibutyl sebacate.

18. The process of claim 12, wherein the active ingredient is selected from the group consisting of omeprazole, pantoprazole, lansoprazole, rabeprazole, duloxetine, or their pharmaceutically acceptable salts such as rabeprazole sodium or their enantiomers such as esomeprazole or pharmaceutically acceptable salts of their enantiomers such as esomeprazole magnesium trihydrate and mixtures thereof.

19. A process as claimed in claim 18 wherein the active ingredient is selected from the group consisting of rabeprazole sodium, esomeprazole and esomeprazole magnesium trihydrate and mixtures thereof.
